(19) United States
(12) Patent Application Publication
(10) Pub. No.: US 2024/0374501 A1
(43) Pub. Date: Nov. 14, 2024

(54) PROCESS FOR TREATING KERATINOUS MATERIAL, COMPRISING THE MIXING OF AT LEAST TWO AGENTS (A) AND (B)

(71) Applicant: Henkel AG & Co. KGaA, Duesseldorf (DE)

(72) Inventors: Constanze Kruck, Grevenbroich (DE); Angela Mueller, Grevenbroich (DE); Gabriele Weser, Essen (DE)

(21) Appl. No.: 18/784,322

(22) Filed: Jul. 25, 2024

Related U.S. Application Data
(63) Continuation of application No. PCT/EP2022/084153, filed on Dec. 2, 2022.

(30) Foreign Application Priority Data
Jan. 26, 2022 (DE) 102022200856.1

(51) Int. Cl. A61K 8/898; A61K 8/86; A61Q 5/10

(52) U.S. Cl. CPC A61K 8/898; A61K 8/86; A61Q 5/10; A61K 2800/43; A61K 2800/88

(57) ABSTRACT

A process for treating keratinous material, comprising providing an agent (a) comprising: at least one amino-functionalized silicone polymer, and at least one cosmetic oil, which is liquid at 20° C. and which is different from, providing an agent (b), comprising water and/or at least alkylene glycol of formula (AG). The method further comprises preparing an application mixture by mixing agents (a) and (b), applying the application mixture to the keratinous material, allowing the application mixture applied to act on the keratinous material, and optionally rinsing the application mixture. A multi-component packaging unit which contains the agents (a) and (b) in two separately fabricated containers is also taught.

### FIELD OF INVENTION

The subject matter of the present application is a process for treating keratinous material, and in particular human hair, which comprises the application of at least two agents (a) and (b). Agent (a) contains at least one amino-functionalized silicone polymer and at least one cosmetic oil which is liquid at 20° C., and agent (b) contains water and/or at least one alkylene glycol. Before application, an application mixture is prepared by mixing agents (a) and (b), which application mixture is applied to the keratinous material, allowed to act, and is optionally washed off again.

A second subject matter of this application is a multi-component packaging unit (kit-of-parts) for dyeing keratinous material, and in particular human hair, which comprises two agents (a) and (b), fabricated separately from one another in two different containers.

### BACKGROUND

Changing the shape and color of keratin material, and in particular human hair, represents an important field of modern cosmetics. To change the hair color, the skilled artisan is familiar with a variety of dyeing systems depending upon the dyeing requirements. Oxidation dyes are typically used for permanent, intense dyeing with good fastness properties and good gray coverage. Such dyes contain oxidation dye precursors, known as developer components and coupler components, which together form the actual dyes under the influence of oxidizing agents such as, for example, hydrogen peroxide. Oxidation dyes are characterized by very long-lasting color results.

When using direct dyes, dyes which are already formed diffuse out of the dyes into the hair fiber. In comparison with oxidative hair dyeing, the colors obtained with direct dyes have a lower durability and a more rapid washing out. Colors with direct dyes usually remain on the hair for a period of between 5 and 20 hair washes.

### SUMMARY OF THE INVENTION

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

The particular advantage of a hair mascara product is that the dyeing compounds, such as pigments, are deposited on the surface of the keratin fibers only in the form of a film. The nature of the keratin fibers themselves itself is thus not changed when the product is applied, so that the application of a hair mascara product is associated with particularly minimal damage to the hair. If the user wishes to return to their original hair color, the coloring can be removed from the keratin fiber again quickly, completely, and without residue, without damaging the fibers or changing the original hair color. The development of pigment-based keratin dyes is therefore fully on trend.

In current works, the problem of the low durability of this coloring system has been addressed. In this context, it was found that the wash fastness of the coloring results obtained with pigments could be greatly improved by combining the pigments with certain amino-functionalized silicone polymers. In addition, the choice of particularly well-suited pigments and pigment concentrations on dark hair achieved a brighter color result so that lightening was even possible with this coloring system, which was possible until now only with oxidative hair treatment agents (bleaching or decolorizing agents).

In addition to these many advantages, however, the coloring system based upon pigments still has some disadvantages. In particular, the fabrication of the aminosilicone has proven to be challenging in this context. When the aminosilicone was incorporated together with pigments into a water-containing or solvent-based formulation, storage instability was observed in many cases, which became optically visible as it became resinous or could also be observed as a formation of many small clumps. This accordingly unstable formulation had a greatly reduced dyeing performance after a certain storage period. For this reason, a transition was made to the use of dyes with pigment and aminosilicone in separately fabricated containers in the hair treatment process. The aminosilicone was provided separately as a concentrate, and this was mixed with a carrier formulation for the hair treatment. Due to the relatively small amount used, on the one hand, the complete transfer of the aminosilicone into the carrier or dyeing formulation was associated with difficulties. In addition, mixing the aminosilicone and carrier or dyeing formulation was also found to be difficult. For example, shaking both components in an application bottle was not possible, since the small amount of aminosilicone could not be homogeneously distributed, and the aminosilicone additionally settled in the applicator tip of the bottle during the shaking. In the case of inhomogeneous mixing of the aminosilicone with the dyeing formulation, the dye result resulting during the dyeing process was correspondingly inhomogeneous.

It was the object of the present invention to provide a dyeing system based upon aminosilicones and pigments, which has good storage stability and produces intensive color results with good fastness properties. In the separate fabrication of the aminosilicone and the carrier formulation, in particular good, rapid, homogeneous, and complete miscibility of the two components is to be ensured, which, after mixing, enables homogeneous and uniform application of the aminosilicone to the hair.

Surprisingly, it has now been found that the aforementioned object can be achieved excellently if keratinous fibers, and in particular hair, has been treated with a ready-to-use agent which was prepared before application by mixing at least two agents (a) and (b). In this case, agent (a) comprises at least one amino-functionalized silicone polymer (a1) and at least one cosmetic oil (a2) which is liquid at 20° C. The oil (a2) serves here to dilute the aminosilicone (a1) to increase the volume of the agent (a) and also to optimize its flow properties. When agent (a) was mixed with a cosmetic carrier formulation (b), it was possible in this way to ensure both as complete a transfer of agent (a) as possible into agent (b) and uniform mixing of both agents (a) and (b). Further agents, e.g., a third agent (c) with pigments, could also be incorporated very well into the mixture of (a) and (b). When the mixture of (a) and (b) (or the mixture of (a) and (b) and (c)) was used on keratin fibers, the aminosilicone was able to be applied very uniformly to the keratin fibers and formed a homogeneous film of uniform thickness there. The work leading to this invention has shown that this method of mixing both in dyes and in other hair treatment agents ensures the uniform application of the aminosilicone.

### DETAILED DESCRIPTION OF THE INVENTION

A first subject matter of the present invention is a process for dyeing keratinous fibers, and in particular human hair, comprising the following steps:
(1) providing an agent (a), the agent (a) containing:
   (a1) at least one amino-functionalized silicone polymer, and
   (a2) at least one cosmetic oil which is liquid at 20° C. and is different from (a1),
(2) providing an agent (b), the agent (b) containing:
   (b1) water and/or at least alkylene glycol of the formula (AG)

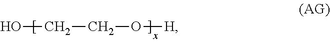

where
x is an integer from 1 to 10,000,
(3) optionally providing an agent (c), the agent (c) containing:
   (c1) at least one pigment,
(4) preparing an application mixture by mixing agents (a) and (b) and optionally (c),
(5) applying the application mixture prepared in step (4) to the keratinous material,
(6) allowing the application mixture applied in step (5) to act upon the keratinous material, and
(7) optionally rinsing out the application mixture.

### Keratinous Fibers

Keratinous fibers are understood to be wool, furs, and feathers, and in particular human hair. Keratinous fibers are very particularly preferably understood to mean human hair.

### Agent for Treating Keratinous Fibers

Agents for treating keratinous fibers are understood to mean, for example, agents for dyeing keratin fibers, agents for reshaping or shaping keratin fibers, or else agents for the conditioning or care of the keratin fibers. The process according to the invention shows particularly good suitability in agents for dyeing keratinous fibers, and in particular human hair.

In the context of this invention, the term, “dyeing agent,” is used for a coloring of the keratin material, and in particular hair, brought about by use of pigments. With this coloring, the pigments are deposited together with the amino silicone (a1) in a particularly homogeneous and uniform film on the surface of the keratin fibers.

### Agent (a)

Agent (a) is provided in step (1) of the process according to the invention. For example, agent (a) can be present in a packaging unit or a container and can thereby be made available to the user. The container can, for example, be a sachet, a bottle, a can, a crucible, or another container suitable for cosmetic formulations. Agent (a) is characterized by its content of at least one amino-functionalized silicone polymer (a1) and at least one cosmetic oil (a2).

### Amino-Functionalized Silicone Polymer (a1) in Agent (a)

Agent (a) contains at least one amino-functionalized silicone polymer as ingredient (a1) essential to the invention. The amino-functionalized silicone polymer can alternatively also be referred to as aminosilicone or amodimethicone.

Silicone polymers are generally macromolecules with a molecular weight of at least 500 g/mol, preferably at least 1,000 g/mol, more preferably at least 2,500 g/mol, and more preferably at least 5,000 g/mol, which comprise repeating organic units.

The maximum molecular weight of the silicone polymer depends upon the degree of polymerization (number of polymerized monomers) and the batch size, and is also determined by the polymerization method. In the context of the present invention, it is preferable if the maximum molecular weight of the silicone polymers is not more than 10⁷ g/mol, preferably not more than 10⁶ g/mol, and particularly preferably not more than 10 ⁵ g/mol.

The silicone polymers comprise many Si—O repeat units, the Si atoms being able to bear organic functional groups such as, for example, alkyl groups or substituted alkyl groups. Alternatively, a silicone polymer is therefore also referred to as polydimethylsiloxane.

Corresponding to the high molecular weight of the silicone polymers, these are based upon more than 10 Si—O repeat units, preferably more than 50 Si—O repeat units and particularly preferably more than 100 Si—O repeat units, and very particularly preferably more than 500 Si—O units.

An amino-functionalized silicone polymer is understood to mean a functionalized silicone which bears at least one structural unit with an amino group. The amino-functionalized silicone polymer preferably bears several structural units with at least one amino group in each instance. An amino group is understood to mean a primary amino group, a secondary amino group, and a tertiary amino group. All these amino groups can be protonated in an acidic environment and are then present in their cationic form.

In principle, it was possible to achieve positive effects with amino-functionalized silicone polymers (a1) when they bore at least one primary, at least one secondary, and/or at least one tertiary amino group. However, intense colorings with the best colorfastness were obtained when an amino-functionalized silicone polymer (a1) containing at least one secondary amino group was used in the agent (a).

In a very particularly preferred embodiment, a process according to the invention is characterized in that agent (a) contains at least one amino-functionalized silicone polymer (a1) with at least one secondary amino group,

The secondary amino groups(s) may be at different positions of the amino-functionalized silicone polymer. Particularly good effects were found when an amino-functionalized silicone polymer (a1) was used that had at least one, and preferably multiple, structural units of the formula (Si-amino).

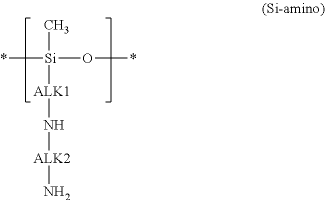

In the structural units of the formula (Si-amino), the abbreviations ALK1 and ALK2 are each independently a linear or branched divalent C₁-C₂₀ alkylene group.

In an additional very particularly preferred embodiment, a process according to the invention is characterized in that the agent (a) contains at least one amino-functionalized silicone polymer (a1) which comprises at least one structural unit of the formula (Si-amino),

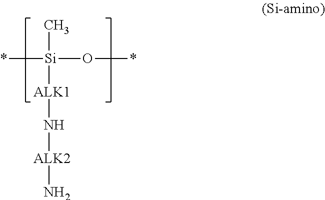

where
ALK1 and ALK2 represent, independently of one another, a linear or branched, divalent C₁-C₂₀ alkylene group.

The positions marked with an asterisk (*) in each case indicate the bond to further structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—), and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In another particularly preferred embodiment, the structural units of the formula (Si-amino) represent repeat units in the amino-functionalized silicone polymer (a1), so that the silicone polymer comprises multiple structural units of the formula (Si-amino).

In the following, particularly well-suited amino-functionalized silicone polymers (a1) with at least one secondary amino group are listed.

Colorings with the very best washing fastnesses were able to be obtained when, in the process according to the invention, an agent (a) was applied to the keratinous material which contains at least one amino-functionalized silicone polymer (a1) that comprises structural units of the formula (Si—I) and formula (Si-II):

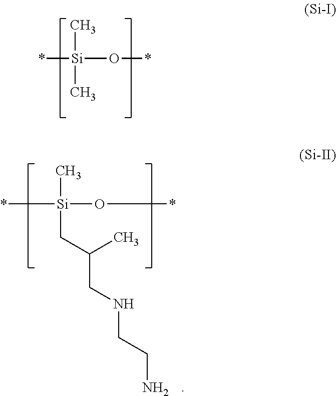

In an additional, explicitly very particularly preferred embodiment, a process according to the invention is characterized in that agent (a) contains at least one amino-functionalized silicone polymer (a1) which comprises structural units of the formula (Si—I) and formula (Si-II):

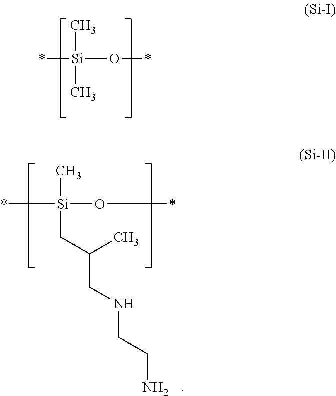

A corresponding amino-functionalized silicone polymer with the structural units (Si—I) and (Si-II) is, for example, the commercial product DC 2-8566 or Dowsil 2-8566 Amino Fluid which is sold commercially by Dow Chemical Company and which bears the designation, “Siloxanes and Silicones, 3-[(2-aminoethyl)amino]-2-methylpropyl Me, Di-Me-Siloxane,” and the CAS number 106842-44-8. A further particularly preferred commercial product is Dowsil AP-8568 Amino Fluid, which is likewise commercially sold by Dow Chemical Company.

In the context of a further preferred embodiment, a process according to the invention is characterized by the application of an agent (a) to the keratinous material, wherein the agent (a) contains at least one amino-functional silicone polymer (a1) of the formula (Si—III),

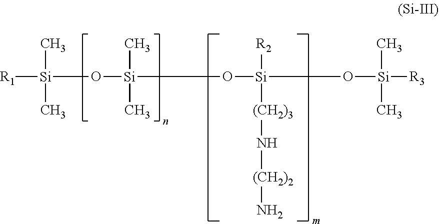

where
m and n denote numbers that are selected such that the sum (n+m) is in a range of 1 to 1,000,
n is a number in a range of 0 to 999, and m is a number in a range of 1 to 1,000,
R1, R2, and R3, which are identical or different, denote a hydroxyl group or a C1-4 alkoxy group,
at least one of the R1 to R3 groups denoting a hydroxyl group.

Further processes preferred according to the invention are characterized by the application of an agent (a) to the keratinous material, wherein the agent (a) contains at least amino-functional silicone polymer (a1) of the formula of the formula (Si—IV),

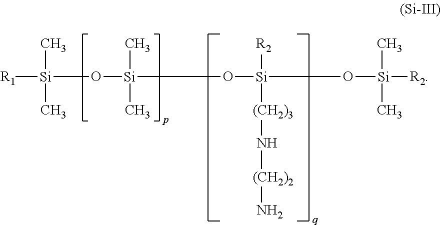

where
p and q denote numbers that are selected such that the sum (p+q) is in a range of 1 to 1,000,
p is a number in a range of 0 to 999, and q is a number in a range of 1 to 1,000,
R1 and R2, which are different, denote a hydroxy group or a C1-4 alkoxy group, at least one of the groups R1 to R2 denoting a hydroxy group.

The silicones of the formulas (Si—III) and (Si—IV) differ by the grouping on the Si atom that carries the nitrogen-containing group: In formula (Si—III), R2 denotes a hydroxy group or a C1-4 alkoxy group, whereas the group in formula (Si—IV) is a methyl group. The individual Si moieties, which are labeled with the indices m and n or p and q, need not be present as blocks; instead, the individual units can also be distributed randomly, i.e., in the formulas (Si—III) and (Si—IV), each R1-Si(CH₃)₂ group is not necessarily bound to a —[O—Si(CH₃)₂] moiety.

Processes according to the invention in which an agent (a) containing at least one amino-functional silicone polymer (a1) of the formula (Si—V) is applied to the keratin fibers have also proven to be particularly effective with regard to the desired effects:

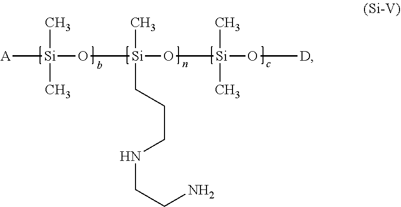

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n, and c stand for integers between 0 and 1,000,
   with the proviso that
      n>0 and b+c>0
      at least one of the conditions A=—OH or D=—H is met.

In the aforementioned formula (Si—V), the individual siloxane units having the indices b, c, and n are randomly distributed, i.e., they are not necessarily block copolymers.

The agent (a) can also comprise one or more different amino-functionalized silicone polymers which are described by the formula (Si—VI),

M(RₐQ_{b}SiO_{4-a-b)/2)x}(R_{c}SiO_{(4-c)2)y}M (Si—VI),
wherein, in the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen, and nitrogen atoms, and Z is an organic, amino-functional group containing at least one amino-functional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, and preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{Z}NH₂, where z is 1 or more. Another possible formula for Z is —NH(CH₂)_{Z}(CH₂)_{ZZ}NH, where both z and zz are independently 1 or more, this structure comprising diamino ring structures such as piperazinyl. Z is most preferably a —NHCH₂CH₂NH₂ functional group. Another possible formula for Z is —N(CH₂)_{Z}(CH₂)_{ZZ}NX₂ or —NX₂, wherein each X of X₂ is selected independently from the group consisting of hydrogen and alkyl groups having 1 to 12 carbon atoms, and zz is 0.

Q is most preferably a polar, amino-functional group of the formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)2} units lies within a range of about 1:2 to 1:65, preferably about 1:5 to about 1:65, and most preferably about 1:15 to about 1:20. If one or more silicones of the above formula are used, then the various variable substituents in the above formula can be different in the various silicone components, present in the silicone mixture.

In the context of a particularly preferred embodiment, a process according to the invention is characterized by the application of an agent (a) to the keratinous material, wherein the agent (a) is an amino-functional silicone polymer of the formula (Si-Vll),

R′ₐG₃₋ₐ-Si(OSiG₂)ₙ-(OSiG_{b}R′_{2-b})ₘ—O-SiG₃₋ₐ-R′ₐ (Si—VII),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
a represents a number between 0 and 3, and in particular 0,
b represents a number between 0 and 1, and in particular 1,
m and n are numbers whose sum (m+n) is between 1 and 2,000, preferably between 50 and 150, n preferably assuming values of 0 to 1,999 and in particular of 49 to 149, and m preferably assuming values of 1 to 2,000, and in particular of 1 to 10,
R′ is a monovalent functional group selected from
   Q-N(R″)—CH₂—CH₂—N(R″)₂
   Q-N(R″)₂
   Q-N⁺(R″)₃A⁻
   Q-N⁺H(R″)₂A⁻
   Q-N⁺H₂(R″)A⁻
   Q-N(R″)—CH₂—CH₂—N⁺R″H₂A⁻,
each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—, R″ represents identical or different functional groups from the group —H, -phenyl, -benzyl, —CH₂—CH(CH₃)Ph, from the C₁₋₂₀ alkyl functional groups—preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃—and A represents an anion preferably selected from chloride, bromide, iodide, or methosulfate.

In the context of a further preferred embodiment, a process according to the invention is characterized by the application of an agent (a) to the keratinous material, wherein the agent (a) contains at least one amino-functional silicone polymer (a1) of the formula (Si-VIIa),

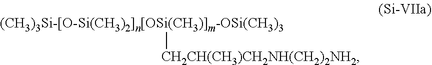

in which m and n are numbers of which the sum (m+n) is between 1 and 2,000, and preferably between 50 and 150, n preferably assuming values of 0 to 1,999 and in particular of 49 to 149 and m preferably assuming values of 1 to 2,000, and in particular from 1 to 10.

These silicones are designated as trimethylsilylamodimethicones in accordance with the INCI Declaration.

In the context of a particularly preferred embodiment, a process according to the invention is characterized by the application of an agent (a) to the keratinous material, wherein the agent (a) contains at least on amino-functional silicone polymer of the formula (Si-VIIb),

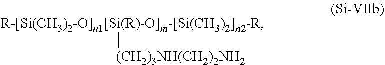

in which R denotes —OH, —O—CH₃, or a —CH₃ group and m, n1, and n2 are numbers whose sum (m+n1+n2) amounts to between 1 and 2,000, and preferably between 50 and 150, the sum (n1+n2) preferably assuming values of 0 to 1,999 and in particular of 49 to 149 and m preferably assuming values of 1 to 2,000, and in particular of 1 to 10.

According to the INCI Declaration, these amino-functionalized silicone polymers are referred to as amodimethicones.

Irrespective of which amino-functional silicones are used, agents (a) according to the invention that contain an amino-functional silicone whose amine value is above 0.25 meq/g, preferably above 0.3 meq/g, and in particular above 0.4 meq/g are preferred. The amine value here represents the milli-equivalents of amine per gram of the amino-functional silicone. Said value can be determined by titration and may also be given in the unit mg KOH/g.

Furthermore, agents (a) which contained a specific 4-morpholinomethyl-substituted silicone polymer (a1) are also suitable for use in the process according to the invention. This amino-functionalized silicone polymer comprises structural units of the formulas (Si—VIII) and of the formula (Si—IX):

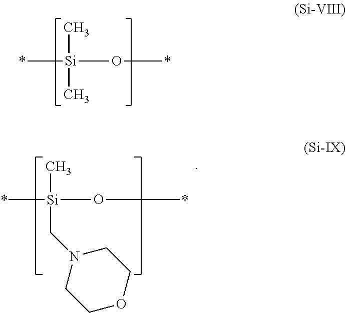

Corresponding 4-morpholinomethyl-substituted silicone polymers are described below.

A very particularly preferred amino-functionalized silicone polymer is known under the name of amodimethicone/morpholinomethyl silsesquioxane copolymer and is commercially available in the form of the raw material Belsil ADM 8301 E from Wacker.

For example, a silicone which has structural units of formulas (Si—VIII), (Si—IX) and (Si—X) can be used as 4-morpholinomethyl-substituted silicone:

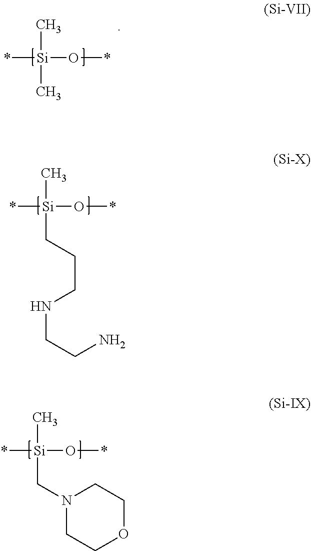

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents (a) according to the invention contain at least one 4-morpholinomethyl-substituted silicone of the formula (Si—XI):

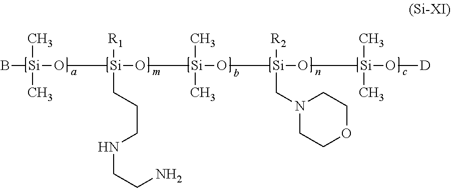

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b, and c represent, independently of one another, integers between 0 and 1,000, with the proviso that a+b+c>0
m and n represent, independently of one another, integers between 1 and 1,000, with the proviso that
   at least one of the conditions B=—OH or D=—H is met,
   the units a, b, c, m, and n are distributed randomly or in blocks in the molecule.

Structural formula (Si—XI) is intended to indicate that the siloxane groups n and m do not necessarily have to be directly bonded to an end group B or D. Instead, in preferred formulas (Si—VI), a>0 or b>0 and, in particularly preferred formulas (Si—VI), a>0 and c>0; i.e., the terminal group B or D is preferably bonded to a dimethylsiloxy group. In formula (Si—VI) as well, the siloxane units a, b, c, m, and n are preferably distributed randomly.

The silicones represented by formula (Si—VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ D=—Si(CH₃)₂OH
and
   These silicones lead to enormous improvements in the hair properties of hair treated with the agents according to the invention, and to greatly improved protection during oxidative treatment.

The amino-functionalized silicone polymers (a1) are preferably used in certain amounts in agent (a). Particularly good miscibility and also good application properties were obtained on the hair when agent (a) contained, relative to the total weight of the agent, one or more amino-functionalized silicone polymers (a1) in a total amount of 1 to 90 wt %, preferably 2 to 70 wt %, more preferably 3 to 50 wt %, still more preferably 4 to 30 wt %, and very particularly preferably 5 to 20 wt %.

In a further explicitly very preferred embodiment, a process according to the invention is characterized in that agent (a) contains, relative to the total weight of the agent (a), one or more amino-functionalized silicone polymers (a1) in a total amount of 1 to 90 wt %, preferably 2 to 70 wt %, more preferably 3 to 50 wt %, still more preferably 4 to 30 wt %, and very particularly preferably 5 to 20 wt %.

### Liquid Cosmetic Oils (a2) in Agent (a)

As a second component (a2) that is essential to the invention, agent (a) contains at least one cosmetic oil which is liquid at 20° C. The oil (a2), which is liquid at 20° C., serves to dilute the aminosilicone (a1), increases the volume of the agent (a), and also improves the flow properties of agent (a). In the context of the present invention, an oil is understood to mean an organic liquid which is liquid or flowable at 20° C. and is not miscible with water. An oil is then not miscible with water if it has a solubility in water of less than 1 wt % at 20° C. (760 mmHg).

The water solubility of the oil can be determined, for example, in the following manner. 1.0 g of the oil is added to a beaker. Water is added up to 100 g. A stirring bar is added, and the mixture is heated to 25° C. on a magnetic stirrer, while stirring. The mixture is stirred for 60 minutes. Thereafter, the aqueous mixture is visually assessed. If a second phase, i.e., a separately present oil phase (e.g., in the form of oil droplets) in addition to the water phase, can also be seen in the oil-water mixture after this period, then the solubility of the oil is less than 1 wt %.

The oil (a2) should improve the miscibility and homogenization of the aminosilico (a1), and therefore it does not itself represent an aminosilicone. For this reason, the oil (a2) is different from the amino-functionalized silicone polymer (a1).

Especially well-incorporated oils (a2) can be selected from the group of non-amino-functionalized oligoalkylsiloxanes, non-amino-functionalized silicone polymers, paraffin oils, isoparaffin oils, synthetic C₃-C₁₂-hydrocarbons, di-C₁₂-C₂₃ alkyl ethers, vegetable oils, and ester oils.

In a further explicitly very particularly preferred embodiment, a process according to the invention is characterized in that agent (a) contains at least one cosmetic oil (a2), which is liquid at 20° C. and is selected from the group of non-amino-functionalized oligoalkylsiloxanes, non-amino-functionalized silicone polymers, paraffin oils, isoparaffin oils, synthetic C₃-C₁₂ hydrocarbons, di-C₁₂-C₂₃ alkyl ethers, vegetable oils, and ester oils.

Particularly well-suited cosmetic oils (a2) are selected from the group of non-amino-functionalized oligoalkylsiloxanes and non-amino-functionalized silicone polymers. In the context of the invention, oligoalkylsiloxanes are understood to mean oligomeric siloxanes, which may be linear or cyclic. The oligoalkylsiloxanes are not amino-functionalized, which means that they do not carry any amino group in their structure.

Preferred linear oligoalkylsiloxanes are compounds of the general formula (OAS-I)

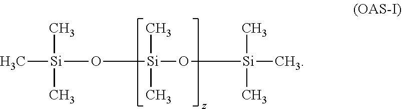

where z represents an integer from 0 to 10. Z preferably represents the numbers 0, 1, 2, or 3.

Very particularly preferred linear oligoalkylsiloxanes are, for example:
hexamethyldisiloxane

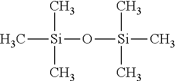

octamethyltrisiloxane

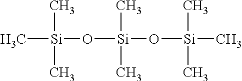

decamethyltetrasiloxane

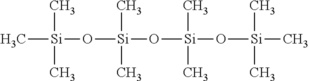

Hexamethyldisiloxane has the CAS number 107-46-0 and can be purchased commercially, for example, from Sigma-Aldrich.

Octamethyltrisiloxane has the CAS number 107-51-7 and is also commercially available from Sigma-Aldrich.

Decamethyltetrasiloxane has the CAS number 141-62-8 and is also commercially available from Sigma-Aldrich.

Preferred cyclic oligoalkylsiloxanes are compounds of the general formula (OAS-II).

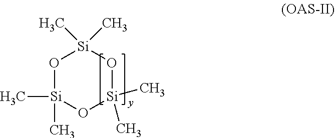

where y represents an integer from 1 to 5. Z preferably represents the numbers 1, 2, or 3.

Very particularly preferred cyclic oligoalkylsiloxanes are, for example, hexamethylcyclotrisiloxane, octamethylcyclotetrasiloxane, and decamethylcyclopentasiloxane.

In a further preferred embodiment, a process according to the invention is characterized in that agent (a) contains at least one cosmetic oil (a2) which is liquid at 20° C. and is selected from the group of oligoalkylsiloxanes of the formula (OAS-I) and/or (OAS-II),

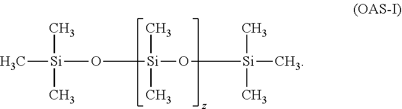

where z represents an integer from 0 to 10, preferably an integer from 0 to 3, and particularly preferably the number 0,

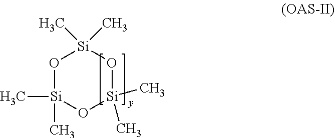

where y represents an integer from 1 to 5, and preferably an integer from 1 to 3.

In another preferred embodiment, a process according to the invention is characterized in that the agent (a) contains a cosmetic oil (a2) which is liquid at 20° C. and which is selected from the group of hexamethyldisiloxane, octamethyltrisiloxane, decamethyltetrasiloxane, hexamethylcyclotrisiloxane, octamethylcyclotetrasiloxane, and/or decamethylcyclopentasiloxane.

The non-amino-functionalized silicone polymers, which are likewise particularly well suited to achieving the object according to the invention, can alternatively also be referred to as silicone oils. The silicone oils are not amino-functionalized, which means that they do not carry an amino group in their structure.

Silicone oils are polymeric compounds whose molecular weight is at least 500 g/mol, preferably at least 1,000 g/mol, more preferably at least 2,500 g/mol, and particularly preferably at least 5,000 g/mol.

The silicone oils comprise Si—O repeat units, wherein the Si atoms are able to bear organic functional groups, such as, for example, alkyl groups or substituted alkyl groups.

Corresponding to the high molecular weight of the silicone oils, these are based upon more than 10 Si—O repeat units, preferably more than 50 Si—O repeat units, and particularly preferably more than 100 Si—O repeat units.

In a further preferred embodiment, a process according to the invention is characterized in that agent (a) contains at least one cosmetic oil (a2), which is liquid at 20° C. and is selected from the group of polydimethylsiloxanes.

It has proven particularly preferable to use in the agent (a) silicone oils, and in particular having a viscosity of 0.5 to 30,000 mm²/s, further preferably of 0.5 to 20,000 mm²/s, even more preferably of 0.5 to 10,000 mm²/s, and very particularly preferably of 0.5 to 500 mm²/s, in each case measured according to ASTM standard D-445.

The ASTM standard D-445 is the standard method for measuring the kinematic viscosity of transparent and opaque fluids.

The viscosity was measured in particular according to ASTM standard D-446, version 06 (D445-06), published June 2006. In this measurement method, the time required for the defined volume of a liquid to flow under defined conditions through the capillary of a calibrated viscometer is measured. With regard to the details of the method, ASMT-D445, refer in particular to ASTM D445-06. Measurement temperature is 25° C. Suitable instruments (such as viscosimeters and thermometers and the corresponding calibrations) are specified in the method.

In the context of a further explicitly very particularly preferred embodiment, a process according to the invention is characterized in that agent (a) contains at least one silicone oil having a viscosity of 0.5 to 30,000 mm²/s, further preferably of 0.5 to 20,000 mm²/s, even more preferably of 0.5 to 10,000 mm²/s, and particularly preferably of 0.5 to 500 mm²/s, measured according to ASTM Standard D-445 (25° C.).

In principle, various silicone oils can be used in agent (c), but the use of polydimethylsiloxanes has proven to be particularly advantageous with regard to the improvement of the feel of the hair and the reduction of its oily texture.

For this reason, it is very particularly preferred if agent (a) contains at least one silicone oil (a2) from the group of polydimethylsiloxanes (dimethicones).

Suitable silicone oils from the group of linear polydimethylsiloxanes are compounds of the general structure (PDMS)

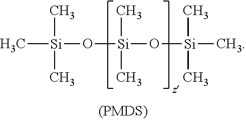

In this case, z′ is selected such that the dimethicones are liquid and preferably have the aforementioned, very particularly well-suited, viscosity ranges.

Preferably, z′ can be an integer from 50 to 100,000, more preferably from 100 to 50,000, and particularly preferably from 500 to 50,000.

Corresponding dimethicones are commercially available from various manufacturers. Very particularly well suited is, for example, the dimethicone which is commercially available under the trade name Xiameters PMX 200 Silicone Fluid 50 CS from Dow Chemicals, the viscosity of which is 50 mm2/s (at 25° C.). This dimethicone is most preferred.

Another particularly well-suited dimethicone is Xiameter PMX 200 Silicone Fluid 100 CS, also available from Dow Corning, the viscosity of which is 100 mm²/s (measurement at 25° C.).

Another particularly well-suited dimethicone is Xiameter PMX 200 Silicone Fluid 350 CS, also available from Dow Corning, the viscosity of which is 350 mm²/s (at 25° C.).

Another particularly well-suited dimethicone is Dow Corning 200 fluid 500 cSt available from Dow Corning, the viscosity of which is 500 mm²/s (at 25° C.).

In a further particularly preferred embodiment, a process according to the invention is characterized in that agent (a) contains at least one cosmetic oil (a2) which is liquid at 20° C. and is selected from the group of polydimethylsiloxanes and which preferably has a viscosity of 0.5 to 30,000 mm²/s, further preferably of 0.5 to 20,000 mm²/s, even more preferably of 0.5 to 10,000 mm²/s, and particularly preferably of 0.5 to 500 mm²/s, measured according to ASTM Standard D-445 (always measured according to ASTM Standard D-445, 25° C.).

Further well-suited oils (a2) are liquid paraffin oils, such as paraffinum liquidum and paraffinum perliquidium, isoparaffin oils, such as isodecane, synthetic hydrocarbons, such as undedane and tridecane and di-n-alkyl ethers having in total between 12 and 36 C atoms, and in particular 12 to 24 C atoms, such as for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether as well as di-tert-butyl ether, diisopentyl ether, di-3-ethyl decyl ether, tert-butyl n-octyl ether, isopentyl n-octyl ether, and 2-methyl pentyl n-octyl ether. The compounds 1,3-di-(2-ethyl-hexyl) cyclohexane (Cetiol® S) and di-n-octyl ether (Cetiol® OE) obtainable as commercial products may be preferred.

Further suitable cosmetic oils (a2) can be selected from the group of vegetable oils. Examples of such oils are sunflower oil, olive oil, soybean oil, rapeseed oil, almond oil, jojoba oil, orange oil, wheat germ oil, peach kernel oil, and the liquid proportions of coconut oil. However, other triglyceride oils, such as the liquid proportions of bovine tallow and synthetic triglyceride oils, are also suitable.

Further suitable cosmetic oils (a2) can also be selected from the group of ester oils. Ester oils are understood to mean the esters of C₆-C₃₀ fatty acids with C₂-C₃₀ fatty alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particularly preferred according to the invention are isotridecyl isononanoate, neopentyl glycol dihepanoate, isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Further suitable cosmetic oils (a2) can be selected from the group of dicarboxylic esters, such as di-n-butyl adipate, di-(2-ethylhexyl) adipate, di-(2-ethylhexyl) succinate and di-isotridecyl acyl, and diol esters, such as ethylene glycol dioleate, ethylene glycol-diisotridecanoate, propylene glycol di(2-ethylhexanoate), propylene glycol diisostearate, propylene glycol dipelargonate, butanediol diisostearate, neopentyl glycol dicaprylate, symmetrical, asymmetrical or cyclic esters of carbonic acid with fatty alcohols, glycerol carbonate, and dicaprylyl carbonate (Cetiol® CC).

The cosmetic oils (a2) are preferably contained in certain quantity ranges in agent (a). Very particularly preferably, agent (a) contains, relative to the total weight of agent (a), one or more cosmetic oils liquid at 20° C. in a total amount of 1 to 99 wt %, preferably 30 to 98 wt %, more preferably 50 to 97 wt %, still more preferably 70 to 96 wt %, and very particularly preferably 80 to 95 wt %.

In the context of another preferred embodiment, a process according to the invention is characterized in that the agent (a) contains, relative to the total weight of the agent (a), one or more cosmetic oils, which are liquid at 20° C., in a total amount of 1 to 99 wt %, preferably 30 to 98 wt %, more preferably of 50 to 97 wt %, still more preferably of 70 to 96 wt %, and very particularly preferably of 80 to 95 wt %.

In the context of another preferred embodiment, a process according to the invention is characterized in that the agent (a) contains, relative to the total weight of the agent (a), one or more non-amino-functionalized oligoalkylsiloxanes in a total amount of 1 to 99 wt %, preferably 30 to 98 wt %, more preferably of 50 to 97 wt %, still more preferably of 70 to 96 wt %, and very particularly preferably of 80 to 95 wt %.

In the context of another preferred embodiment, a process according to the invention is characterized in that the agent (a) contains, relative to the total weight of the agent (a), one or more silicone oils from the group of dimethicones in a total amount of 1 to 99 wt %, preferably 30 to 98 wt %, more preferably of 50 to 97 wt %, still more preferably of 70 to 96 wt %, and very particularly preferably of 80 to 95 wt %.

### Ratios of (a1) to (a2) in Agent (a)

To optimize the performance properties, agent (a) contains components (a1) and (a2) particularly preferably in certain weight ratios relative to one another. In the work leading to this invention, it was observed that the adjustment of the weight ratio from the liquid oils (a2) contained in the agent to the amnosilicones (a1) contained in the agent (a) resulted in the best possible mixing, good storage stability, and very good results in a subsequent coloring.

Particularly good results were obtained if agent (a) contained one or more cosmetic oils (a2) and one or more amino-functionalized silicone polymers (a1), i.e., components (a2)/(a1), in a weight ratio of 100:1 to 1:100, preferably of 100:1 to 1:1, more preferably of 50:1 to 2:1, even more preferably of 40:1 to 4:1, and very particularly preferably of 20:1 to 4:1.

In a further particularly preferred embodiment, a process according to the invention is characterized in that agent (a) contains the cosmetic oils (a2) and the amino-functionalized silicone polymers (a1), i.e., components (a2)/(a1), in a weight ratio of 100:1 to 1:100, preferably of 100:1 to 1:1, more preferably of 50:1 to 2:1, even more preferably of 40:1 to 4:1, and very particularly preferably of 20:1 to 4:1.

In other words, it could be found that both very good mixing and homogeneous coloring were achieved if agent (a) contained the cosmetic oils (a2), in comparison with the aminosilicones (a1), in a 4-fold to 20-fold weight excess.

### Agent (b)

In step (2) of the process according to the invention, a cosmetic agent (b) is provided. Like agent (a), agent (b) can also preferably be present to the user in a packaging unit or a container and can thereby be made available to the user. The container can, for example, be a sachet, a bottle, a can, a crucible, or another container suitable for cosmetic formulations.

The agent (b) represents a cosmetic carrier formulation, and is characterized in that this cosmetic carrier is either water or an alkylene glycol of the formula (AG),

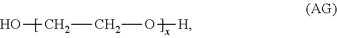

where
x is an integer from 1 to 10,000.

A mixture of water and alkylene glycol is also suitable as a cosmetic carrier, and in this case the agent (b) contains both water and alkylene glycol of the formula (AG).

The alkylene glycols of formula (AG) are protic substances having at least two hydroxyl groups which, due to their repeating —CH₂—CH₂—O—unit, can also be referred to as polyethylene glycols insofar as x is a value of at least 2. In the alkylene glycols (b1) of the formula (AG), x is an integer from 1 to 10,000. In the context of the work leading to this invention, it was found that these polyethylene glycols exhibit particularly favorable suitability for, on the one hand, improving the fastness properties of the dyes and, on the other, also optimally adjusting the viscosity of the agents.

Depending upon their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C. and therefore a pasty consistency. With molecular masses above 3,000 g/mol, the PEG's are solid substances and are commercially available as flakes or powders.

Especially the use of low molecular weight alkylene glycols (or, respectively, polyethylene glycols) has proven to be well suited for achieving the object according to the invention. In the event of low molecular weight alkylene glycols (or polyethylene glycols) in the context of the present invention, x denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

In the context of another very particularly preferred embodiment, a process according to the invention is characterized in that the agent (b) contains:
(b1) at least one alkylene glycol of the formula (AG-1), where

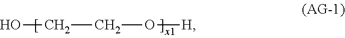

x1 is an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9, and PEG-10.

Another well-suited polyethylene glycol is PEG-32, for example. PEG-32 comprises 32 ethylene glycol units (x1=32), has a mean molar mass of 1,500 g/mol, and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols in the sense of the present invention can be represented by the formula (AG-2), the index number x2 standing for an integer from 101 to 10,000.

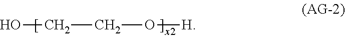

In the case of very well-suited high molecular weight polyethylene glycols, x2 denotes an integer from 101 to 1,000, preferably an integer from 105 to 800, more preferably an integer from 107 to 600, even more preferably an integer from 109 to 400, and very particularly preferably an integer from 110 to 200.

In the context of another very particularly preferred embodiment, a process according to the invention is characterized in that agent (b) contains:
(b1) at least one alkylene glycol of the formula (AG-2), where

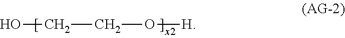

x2 denotes an integer from 101 to 1,000, preferably an integer from 105 to 800, more preferably an integer from 107 to 600, even more preferably an integer from 109 to 400, and very particularly preferably an integer from 110 to 200.

A very particularly well-suited high molecular weight polyethylene glycol is, for example, PEG 6000, which can be obtained commercially from the National Starch company (China). The molecular weight of PEG 6000 is 6,000 to 7,500 g/mol, corresponding to an x2 value of 136 to 171.

Another well-suited polyethylene glycol is PEG 12000 which, for example, is commercially sold by CG chemicals under the trade name of polyethylene glycol 12000 S (or PEG 12000 S). The molecular weight of PEG 12000 is given at 10,500 to 15,000 g/mol, corresponding to an x2 value of 238 to 341.

Another well-suited polyethylene glycol is also PEG 20000 which is commercially available under the trade name polyglycol 20000 P or under the alternative name PEG-350 from Clariant. For PEG 20000, an average molecular weight of 20,000 g/mol is given, which corresponds to an x2 value of 454.

Surprisingly, it has been found that dyes which contain both a low molecular weight polyethylene glycol and a high molecular weight polyethylene glycol have particularly favorable application properties, since these agents have both very good fastness properties and are optimized with regard to their rheological profile.

In the context of another explicitly very particularly preferred embodiment, a process according to the invention is characterized in that the agent (b) contains:
(b11) at least one first alkylene glycol of the formula (AG-1), where

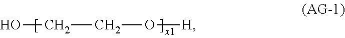

x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(b12) at least one second alkylene glycol of the formula (AG-2), wherein

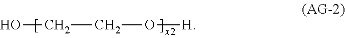

x2 denotes an integer from 101 to 1,000, preferably an integer from 105 to 800, more preferably an integer from 107 to 600, even more preferably an integer from 109 to 400, and very particularly preferably an integer from 110 to 200.

To further optimize the application properties, the agent (b) contains the alkylene glycol(s) (b1) of the formula (AG) preferably in certain quantity ranges which, for example—relative to the total weight of the agent—can be in the range of 10.0 to 99.0 wt %, preferably 30.0 to 95.0 wt %, more preferably 40.0 to 90.0 wt %, and very particularly preferably 40.0 to 80.0 wt %.

In the context of another particularly preferred embodiment, a process according to the invention is characterized in that agent (b) contains, relative to the total weight of agent (b), one or more alkylene glycols (b1) of the formula (AG) in a total amount of 10.0 to 99.0 wt %, preferably of 30.0 to 95.0 wt %, further preferably of 40.0 to 90.0 wt %, and very particularly preferably of 40.0 to 80.0 wt %.

In another particularly preferred embodiment, a process according to the invention is characterized in that the agent (b) contains, relative to the total weight of the agent (b), one or more alkylene glycols (b1) of the formula (AG-1) and/or of the formula (AG-2) in a total amount of 10.0 to 99.0 wt %, preferably 30.0 to 95.0 wt %, more preferably 40.0 to 90.0 wt %, and very particularly preferably 40.0 to 80.0 wt %.

### Water Content in Agent (b)

Agent (b) can also be a water-based agent that contains water in addition to or instead of the alkylene glycols. Agent (b) preferably has a low to moderate water content. It has been found that particularly those agents are well-suited which contain, relative to the total weight of the agent, 0.1 to 50.0 wt %, preferably 0.5 to 35.0 wt %, more preferably 1.0 to 20.0 wt %, and particularly preferably 1.5 to 15.0 wt % water.

In another explicit embodiment, a process according to the present invention is characterized in that the agent (b) contains, relative to the total weight of the agent (b), 0.1 to 50.0 wt %, preferably 0.5 to 35.0 wt %, more preferably 0.1 to 20.0 wt %, and especially preferably 1.5 to 15.0 wt % water.

### Use of Pigments in the Process for Treating Keratin Fibers

In principle, the process according to the invention is suitable for various processes for treating keratin fibers or hair. These can be all the processes in which a uniform application of the aminosilicone (a1) is of particular importance, such as coloring, hair straightening, or permanent wave. In the case of hair coloring based upon pigments, the uneven application of aminosilicone to the hair results in an uneven color result and is therefore particularly strongly visible to the user. For this reason, it is explicitly very particularly preferred if the process according to the invention is a pigment-based dyeing process.

When agents (a) and (b) are applied in a dyeing process, various embodiments have been found to be particularly well suited. In the context of this embodiment, one or more pigments can be incorporated directly into the agent (b), so that agent (b) can also be referred to as a dye.

In the context of a further particularly preferred embodiment, a process according to the invention is characterized in that it is a process for dyeing keratinous material, and in particular human hair, and that the agent (b) contains:
(b2) at least one pigment.

In the context of this embodiment, the ready-to-use dye is prepared by mixing agents (a) and (b).

Therefore, a process for dyeing keratinous fibers, and in particular human hair, comprising the following steps is particularly preferred (renumbered accordingly):
(1) providing an agent (a), the agent (a) containing: (a1) at least one amino-functionalized silicone polymer, and (a2) at least one cosmetic oil which is liquid at 20° C. and is different from (a1),
(2) providing an agent (b), the agent (b) containing:
   (b1) water and/or at least alkylene glycol of the formula (AG)

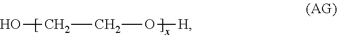

where
x represents an integer from 1 to 10,000, and
(b2) at least one pigment,
(3) preparing an application mixture by mixing the agents (a) and (b),
(4) applying the application mixture prepared in step (3) to the keratinous material,
(5) allowing the application mixture applied in step (4) to act upon the keratinous material, and
(6) optionally rinsing out the application mixture.

Certain pigments, such as, in particular, metal pigments or luster pigments, have sometimes proven to be unstable in aqueous solution. Since agent (b), as described above, preferably has a certain proportion of water, sensitive pigments can corrode in this agent. To avoid these incompatibilities, the pigments can therefore also be fabricated in a further separate agent (c).

In the context of a further particularly preferred embodiment, a process according to the invention is characterized in that it is a process for dyeing keratinous material, and in particular human hair, and further characterized by
(3) providing an agent (c), the agent (c) containing:
   (c1) at least one pigment, and
(4) preparing an application mixture by mixing agents (a) and (b) and (c).

In the context of this embodiment, the application-related dye is prepared by mixing agents (a) and (b) and (c).

The invention therefore further very particularly preferably relates to a process for dyeing keratinous fibers, and in particular human hair, comprising the following steps:
(1) providing an agent (a), agent (a) containing:
   (a1) at least one amino-functionalized silicone polymer, and
   (a2) at least one cosmetic oil which is liquid at 20° C. and is different from (a1),
(2) providing an agent (b), the agent (b) containing:
   (b1) water and/or at least alkylene glycol of the formula (AG)

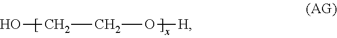

where
x is an integer from 1 to 10,000,
(3) providing an agent (c), the agent (c) containing:
   (c1) at least one pigment,
(4) preparing an application mixture by mixing agents (a) and (b) and (c),
(5) applying the application mixture prepared in step (4) to the keratinous material,
(6) allowing the application mixture applied in step (5) to act upon the keratinous material, and
(7) optionally rinsing out the application mixture.

When pigment mixtures are used, it is likewise possible if a first pigment or a first pigment mixture is used in the agent (b), and a second pigment or a second pigment mixture is used in the agent (c).

Pigments within the meaning of the present invention are understood to mean dyeing compounds which have a solubility of less than 0.5 g/L, preferably of less than 0.1 g/L, and even more preferably of less than 0.05 g/L at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable dye pigments may be of inorganic and/or organic origin.

In a preferred embodiment, an agent (b) according to the invention is characterized in that it contains at least one dyeing compound (b3) from the group of inorganic and/or organic pigments.

Preferred color pigments are selected from synthetic or natural inorganic pigments. Inorganic color pigments of natural origin can be produced, for example, from chalk, ocher, umbra, green soil, burnt Sienna, or graphite. Furthermore, black pigments such as, for example, iron oxide black, chromatic pigments such as, for example, ultramarine or iron oxide red, and also fluorescent or phosphorescent pigments can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, Cl 77007, Pigment Blue 29), chromium oxide hydrate (CI 77289), Iron Blue (ferric ferrocyanide, Cl77510) and/or carmine (cochineal).

Color pigments which are likewise particularly preferred according to the invention are colored pearlescent pigments. These are usually based upon mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica—primarily muscovite or phlogopite—is coated with a metal oxide.

As an alternative to natural mica, synthetic mica coated with one or more metal oxides(s) can also be used as a pearlescent pigment. Particularly preferred pearlescent pigments are based upon natural or synthetic mica and are coated with one or more of the aforementioned metal oxides. The color of the respective pigments can be varied by varying the layer thickness of the metal oxide(s).

In a further preferred embodiment, a process according to the invention is characterized in that agent (b) and/or agent (c) contains at least one pigment from the group of inorganic pigments preferably selected from the group of colored metal oxides, metal hydroxides, metal oxide hydrates, silicates, metal sulfides, complex metal cyanides, metal sulphates, bronze pigments and/or from mica-based colored pigments, which are coated with at least one metal oxide and/or one metal oxychloride.

Examples of particularly suitable color pigments are commercially available, for example, under the trade names Rona®, Colorona®, Xirona®, Dichrona®, and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (titanium dioxide), Silica, CI 77491 (iron oxides), tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (iron oxides), Mica, CI 77891 (titanium dioxide)
Colorona Bright Gold, Merck, Mica, CI 77891 (titanium dioxide), CI 77491 (iron oxides)
Colorona Blackstar Gold, Merck, MICA, CI 77499 (IRON OXIDES)

Additional particularly preferred color pigments with the trade name
Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name
Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

The organic pigments according to the invention are correspondingly insoluble, organic dyes or color varnishes, which may be selected, for example, from the group of nitroso, nitro, azo, xanthene, anthraquinone, isoindolinone, isoindoline, quinacridone, perinone, perylene, diketopyrrolopyrrole, indigo, thioindido, dioxazine, and/or triarylmethane compounds.

Particularly well-suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In another particularly preferred embodiment, a process according to the invention is characterized in that agent (b) and/or agent (c) contains at least one pigment from the group of organic pigments, preferably selected from the group of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

The organic pigment can also be a color lacquer. The term, color lacquer, in the sense of the invention is understood to mean particles which comprise a layer of absorbed dyes, with the unit consisting of particles and dye being insoluble under the above-mentioned conditions. The particles may, for example, be inorganic substrates which may be aluminum, silica, calcium borosilicate, calcium aluminum borosilicate, or also aluminum.

For example, the alizarin color lacquer can be used as the color lacquer.

For the dyeing of the keratin fibers, pigments of a specific shape may also have been used. For example, a pigment based upon a lamellar and/or lenticular substrate platelet may be used. Furthermore, the dyeing is also possible based upon a small substrate plate which comprises a vacuum-metalized pigment.

In the context of another preferred embodiment, a process according to the invention is characterized in that agent (b) and/or agent (c) also contains at least one pigment selected from the group of pigments based upon a lamellar substrate plate, pigments based upon a lenticular substrate plate, and vacuum-metalized pigments.

The small substrate plates of this type have an average thickness of at most 50 nm, preferably less than 30 nm, and particularly preferably at most 25 nm—for example, at most 20 nm. The average thickness of the small substrate plates is at least 1 nm, preferably at least 2.5 nm, and particularly preferably at least 5 nm—for example, at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm, and 10 to 20 nm. Preferably, each substrate plate has as uniform a thickness as possible.

Due to the small thickness of the small substrate plates, the pigment has a particularly high covering power.

The small substrate plates have a preferably monolithic structure. Monolithic in this context means consisting of a single, self-contained unit without fractures, stratifications, or inclusions, although structural changes may, however, occur within the small substrate plates. The substrate plates are preferably composed homogeneously, i.e., there is no concentration gradient within the plates. In particular, the small substrate plates are not constructed in layers and have no particles distributed therein.

The size of the substrate plate can be matched to the respective application, and in particular to the desired effect on the keratin material. In general, the small substrate plates have an average largest diameter of approximately 2 to 200 μm, and in particular approximately 5 to 100 μm.

In a preferred embodiment, the form factor (aspect ratio), expressed by the ratio of the average size to the average thickness, is at least 80, preferably at least 200, more preferably at least 500, and particularly preferably more than 750. In this case, the average size of the uncoated small substrate plates is understood to mean the d50 value of the uncoated small substrate plates. Unless stated otherwise, the d50 value was determined using a device of the Sympatec Heios type, with Quixel wet dispersion. To prepare the sample, the sample to be investigated was pre-dispersed in isopropanol for a period of 3 minutes.

The small substrate plates may be constructed from any material that can be made into the form of a small plate.

They can be of natural origin, but can also be produced synthetically. Materials from which the small substrate plates can be constructed are, for example, metals and metal alloys, metal oxides, and preferably aluminum oxide, inorganic compounds, and minerals such as mica and (semi-)precious stones, as well as plastics. Preferably, the small substrate plates are made of metal (alloy)s.

Any metal suitable for metallic luster pigments is suitable as the metal. Such metals are, inter alia, iron and steel, and all air-resistant and water-resistant (semi-)metals such as, for example, platinum, zinc, chromium, molybdenum, and silicon, as well as alloys thereof such as aluminum bronzes and brass. Preferred metals are aluminum, copper, silver, and gold. Preferred small substrate plates are small aluminum plates and small brass plates, small substrate plates made of aluminum being particularly preferred.

Lamellar small substrate plates are characterized by an irregularly structured edge, and are also referred to as “cornflakes” due to their appearance.

Due to their irregular structure, pigments based upon lamellar small substrate plates produce a high fraction of scattered light. In addition, the pigments based upon lamellar small substrate plates do not completely cover the existing color of a keratin material and, for example, effects can be achieved analogous to a natural graying.

Small lenticular (=lens-shaped) substrate plates have a substantially regular round edge and are also referred to as “silver dollars” due to their appearance. Due to their regular structure, the fraction of the reflected light predominates in pigments based upon small lenticular substrate plates.

Vacuum-metallized pigments (VMP) can be obtained, for example, by releasing metals, metal alloys, or metal oxides from correspondingly coated films. These are characterized by a particularly small thickness of the small substrate plates in the range of 5 to 50 nm and by a particularly smooth surface having increased reflectivity. Substrate plates which comprise a pigment metallized in a vacuum are also referred to as VMP substrate plates in the context of this application. VMP substrate plates of aluminum can be obtained, for example, by releasing aluminum from metallized films.

The small substrate plates made of metal or metal alloy can be passivated, for example by anodizing (oxide layer) or chromatizing.

Uncoated lamellar, lenticular, and/or VPM substrate platelets, and in particular those made of metal or metal alloy, reflect the incident light to a high degree and produce a light-dark flop. These have proven to be particularly preferred for use in the dye.

Suitable pigments based upon a lamellar substrate platelet include, for example, the pigments of the VISIONAIRE series by Eckart.

Pigments based upon a lenticular substrate platelet are available, for example, under the name Alegrace® Gorgeous from the company Schlenk Metallic Pigments GmbH.

Pigments based upon a small substrate plate, which comprises a vacuum-metallized pigment, are available, for example, under the name Alegrace® Marvelous or Alegrace® Aurous from the company Schlenk Metallic Pigments GmbH.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ of 1.0 to 50 μm, preferably of 5.0 to 45 μm, preferably of 10 to 40 μm, and in particular of 14 to 30 μm. The mean particle size D₅₀ can be determined, for example, using dynamic light scattering (DLS).

It is further preferred if the pigments used have a certain particle size. It is therefore advantageous according to the invention if the at least one pigment has a mean particle size D₅₀ from 1.0 to 50 μm, preferably from 5.0 to 45 μm, preferably from 10 to 40 μm, and in particular from 14 to 30 μm. The average particle size D₅₀ can example

The pigment or pigments are used very particularly preferably in certain quantity ranges in agent (b) and/or in agent (c). Particularly positive results were obtained when agent (b) and/or agent (c) contained, relative to the total weight of the respective agent, one or more pigments in a total amount of 0.05 to 10.0 wt %, preferably 0.1 to 7.0 wt %, more preferably 0.2 to 5.0 wt %, and very particularly preferably 0.3 to 3.0 wt %.

In another very particularly preferred embodiment, a process according to the invention is characterized in that agent (b) and/or agent (c) contains, relative to the total weight of the respective agent, one or more pigments in a total amount of 0.05 to 10.0 wt %, preferably 0.1 to 7.0 wt %, more preferably 0.2 to 5.0 wt %, and very particularly preferably 0.3 to 3.0 wt %.

### Addition Products of C₁-C₆ Alkylene Oxide(s) to the Esters of C₁₂-C₃₀ Fatty Acids and Aromatic C₁-C₁₂ Alcohols (a5)

To further improve the fastness properties, such as rub fastness and wash fastness, the agent according to the invention may additionally contain, as an optional component, at least one addition product of C₁-C₆ alkylene oxide(s) to the esters of C₁₂-C₃₀ fatty acids and aromatic C₁-C₁₂ alcohols, which fall under compounds of the general formula (AFE-I)

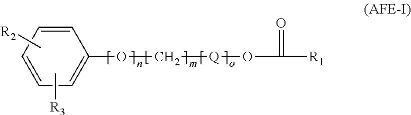

where
R1 represents a saturated or unsaturated C₁₁-C₂₉ alkyl group,
R2, R3 represent, independently of one another, a hydrogen atom, a C₁-C₆ alkyl group, a hydroxy group, or a C₁-C₆ alkoxy group,
n represents the number 0 or 1,
m represents an integer from 0 to 6,
represents an integer from 1 to 60, and
Q represents a structural unit —O—CH₂—CH₂—, —O—CH(CH₃)—CH₂—, or —O—CH₂—CH(CH₃)—,
with the proviso that if m is equal to 0, n is also equal to 0.

In the context of another particularly preferred embodiment, a process according to the invention is characterized in that agent (b) contains:
(b3) at least one alkoxylated fatty acid ester of general formula (AFE-I)

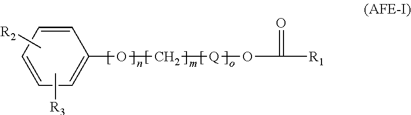

where
R1 represents a saturated or unsaturated C₁₁-C₂₉ alkyl group,
R2, R3 represent, independently of one another, a hydrogen atom, a C₁-C₆ alkyl group, a hydroxy group, or a C₁-C₆ alkoxy group,
n represents the number 0 or 1,
m represents an integer from 0 to 6,
represents an integer from 1 to 60, and
Q represents a structural unit —O—CH₂—CH₂—, —O—CH(CH₃)—CH₂—, or —O—CH₂—CH(CH₃)—,
with the proviso that if m is equal to 0, n is also equal to 0.

The functional group R1 represents a saturated or unsaturated C₁₁-C₂₉ alkyl group. An unsaturated C₁₁-C₂₃ alkyl group can comprise one or more double bonds and is alternatively also termed an unsaturated C₁₁-C₂₃ alkenyl group. The saturated or unsaturated C₁₁-C₂₉ alkyl group can be linear or branched.

Preferably, R1 represents a linear, saturated or unsaturated C₁₁-C₂₃ alkyl group.

The functional groups R2 and R3 are each a hydrogen atom, a C₁-C₆ alkyl group, a hydroxy group, or a C₁-C₆ alkoxy group. Very particularly preferably, the functional groups R2 and R3 both stand for a hydrogen atom.

The index number n is the number 0 or 1. Preferably, n is the number 0.

The index number m is an integer from 0 to 6. Preferably, m is the number 1.

The index number o is an integer from 1 to 60. Preferably, o is an integer from 1 to 30, more preferably 1 to 20, even more preferably 1 to 10, and very particularly preferably 1 to 5.

The functional group Q is a structural unit —O—CH₂—CH₂—, —O—CH(CH₃)—CH₂—, or —O—CH₂—CH(CH₃)—. Particularly preferably, Q is a structural unit —O—CH(CH₃)—CH₂— or —O—CH₂—CH(CH₃)—.

If o is a number greater than 1, several structural units Q are present in the compounds of formula (AFE-I) (or also of formula (AFE-II)), in which case each structural unit Q can be selected independently of the other structural units Q.

In the context of another very particularly preferred embodiment, an agent according to the invention is characterized in that it contains:
(b3) at least one alkoxylated fatty acid ester of general formula (AFE-I)

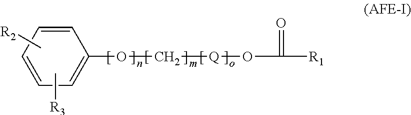

where
R1 represents a saturated or unsaturated C₁₁-C₂₉ alkyl group
R2, R3 are both are a hydrogen atom,
n is the number 0,
m is the number 1,
is an integer 1 to 30, preferably 1 to 20, more preferably 1 to 10, and very particularly preferably 1 to 5, and
Q is a structural unit —O—CH(CH₃)—CH₂— or —O—CH₂—CH(CH₃)—.

An explicitly particularly well-suited compound of this type is PPG-3 benzyl ether myristate, which is alternatively also referred to as α-(1-oxotetradecyl)-ω-(phenylmethoxy) poly [oxy(methyl-1,2-ethanediyl)] and bears the CAS number 642443-86-5.

PPG-3 benzyl ether myristate can be purchased commercially, for example, under the trade name of Crodamol STS from Croda.

In the context of a further, very particularly preferred embodiment, a process according to the invention is characterized in that agent (b) contains PPG-3 benzyl ether myristate.

The alkoxylated fatty acid esters (b3) are particularly preferably used within specific quantity ranges in the agent according to the invention. Particularly good results were obtained when the agent (b) contained, relative to the total weight of the agent (b), one or more alkoxylated fatty acid esters (b3) in a total amount of 0.1 to 20.0 wt %, preferably 0.5 to 15.0 wt %, more preferably 1.0 to 10.0 wt %, still more preferably 1.0 to 8.0 wt %, and very particularly preferably 1.0 to 5.0 wt %.

Addition Products of C₁-C₆ Alkylene Oxide(s) to Aliphatic C₁-C₂₄ Alkanols

To further improve the fastness properties, such as rub fastness and wash fastness, the agent (b) according to the invention may additionally contain, as an optional component, at least one addition product of C₁-C₆ alkylene oxides to C₁-C₂₄ alkanols.

In the context of another very particularly preferred embodiment, a process according to the invention is therefore also characterized in that agent (b) contains:
(b4) at least one addition product of C₁-C₆ alkylene oxide(s) to aliphatic C₁-C₂₄ alkanols.

The further optionally contained components (b4) can be used in addition to the ingredients of group (b3). However, the further optionally contained components (b4) can also be used instead of the ingredients of group (b3).

C₁-C₆ alkylene oxides suitable according to the invention and their preferred and particularly preferred representatives have already been defined in the preceding sections.

The C₁-C₂₄ alkanols are, in accordance with the invention, compounds having 1 to 14 carbon atoms and a hydroxyl group. The following can be mentioned by way of example: methanol, ethanol, 1-propanol, 2-propanol, 1-butanol, 2-butanol, 1-pentanol, 2-pentanol, 3-pentanol, 1-hexanol, 2-hexanol, 3-hexanol, 1-heptanol, 2-heptanol, 3-heptanol, 4-heptanol, 1-octanol, 2-octanol, 3-octanol, 4-octanol, 1-nonanol, 2-nonanol, 3-nonanol, 4-nonanol, 5-nonanol, 1-decanol, 2-decanol, 3-decanol, 4-decanol, 5-decanol, 1-undecanol, 2-undecanol, 3-undecanol, 4-undecanol, 5-undecanol, 6-undecanol, 1-dodecanol, 2-dodecanol, 3-dodecanol, 4-dodecanol, 5-dodecanol, 6-dodecanol, 1-tridecanol, 2-tridecanol, 3-tridecanol, 4-tridecanol, 5-tridecanol, 6-tridecanol, 7-tridecanol, 1-tetradecanol, 2-tetradecanol, 3-tetradecanol, 4-tetradecanol, 5-tetradecanol, 6-tetradecanol, 7-tetradecanol, 1-pentadecanol, 2-pentadecanol, 3-pentadecanol, 4-pentadecanol, 5-pentadecanol, 6-pentadecanol, 7-pentadecanol, 8-pentadecanol, 1-hexadecanol, 2-hexadecanol, 3-hexadecanol, 4-hexadecanol, 5-hexadecanol, 6-hexadecanol, 7-hexadecanol, 8-hexadecanol, 1-heptadecanol, 2-heptadecanol, 3-heptadecanol, 4-heptadecanol, 5-heptadecanol, 6-heptadecanol, 7-heptadecanol, 8-heptadecanol, 1-octadecanol, 2-octadecanol, 3-octadecanol, 4-octadecanol, 5-octadecanol, 6-octadecanol, 7-octadecanol, 8-octadecanol, and 9-octadecanol.

Particularly well-suited addition products of C₁-C₆ alkylene oxide(s) to aliphatic C₁-C₂₄ alkanols (b4) are the compounds of general formula (AA-I)

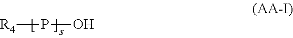

wherein
R4 represents a saturated or unsaturated C₁-C₂₄ alkyl group, and
P represents a structural unit —O—CH₂—CH₂—, —O—CH(CH₃)—CH₂—, or —O—CH₂—CH(CH₃)—, and
s represents an integer from 1 to 60.

The functional group R4 represents a saturated or unsaturated C₁-C₂₄ alkyl group. The functional groups R4 can be unsaturated as of a carbon number of at least 2 C atoms. An unsaturated C₂-C₂₄ alkyl group can comprise one or more double bonds and is alternatively also referred to as an unsaturated C₂-C₂₄ alkenyl group. The saturated or unsaturated C₁-C₂₄ alkyl group can be linear or branched.

Particularly preferably, the functional group R4 is a saturated, unbranched C₁-C₁₂ alkyl group. Very particularly preferably, the functional group R4 is a saturated, unbranched C₁-C₆ alkyl group.

In the compounds of formula (AA-I), P represents a structural unit —O—CH₂—CH₂—, —O—CH(CH₃)—CH₂—, or —O—CH₂—CH(CH₃)—. The number of structural units contained in the compounds of the formula (AA-I) is obtained by the index number s. In this case, the structural units P are oriented such that the oxygen atom in each group —O—CH₂—CH₂—, —O—CH(CH₃)—CH₂—, and —O—CH₂—CH(CH₃)—is adjacent to the alkyl group R4, and the relevant unit —CH₂— or —CH(CH₃)—borders the hydroxy group —OH.

If s is a number greater than 1, several structural units P are present in the compounds of formula (AA-I). In this case, each structural unit P can be selected independently of the other structural units P.

The index number s represents an integer from 1 to 60, preferably an integer from 1 to 40, further preferably an integer from 10 to 30, and very particularly preferably an integer from 10 to 20.

In the context of another particularly preferred embodiment, a process according to the invention is characterized in that agent (b) contains:
(b4) at least one addition product of C₁-C₆ alkylene oxide(s) to aliphatic C₁-C₂₄ alkanols of the formula (AA-I),

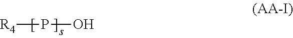

wherein
R4 represents a saturated or unsaturated C₁-C₂₄ alkyl group, preferably a C₁-C₁₂ alkyl group, and particularly preferably a C₁-C₆ alkyl group, and
P represents a structural unit —O—CH₂—CH₂—, —O—CH(CH₃)—CH₂—, or —O—CH₂—CH(CH₃)—, and
s represents an integer from 1 to 60, preferably an integer from 1 to 40, further preferably an integer from 10 to 30, and very particularly preferably an integer from 10 to 20.

A very particularly well-suited addition product of C₁-C₆ alkylene oxide(s) to aliphatic C₁-C₂₄ alkanols of the formula (AA-I) is propylene glycol monobutyl ether, which is also referred to as PPG-14 butyl ether and bears the CAS number 9003-13-8. PPG-14 butyl ether can be purchased commercially under the trade name Ucon Fluid AP from Dow.

In the context of a further very particularly preferred embodiment, a process according to the invention is therefore further characterized in that the agent (b) contains PPG-14 butyl ether.

The alkoxylated alkanols (b4) are particularly preferably used within specific quantity ranges in the agent according to the invention.

Particularly good results were obtained when agent (b) contained, relative to the total weight of agent (b), one or more alkoxylated alkanols (b4) in a total amount of 0.1 to 20.0 wt %, preferably 0.2 to 15.0 wt %, more preferably 0.3 to 10.0 wt %, still more preferably 0.4 to 5.0 wt %, and very particularly preferably 0.5 to 3.0 wt %.

### Further Optional Ingredients in Agents (a), (b), and (c)

In addition to the above-described and optional components that are essential to the invention, agents (a) and/or (b) and/or (c) can also contain further optional ingredients.

The agents can also contain other active ingredients, auxiliaries, and additives, e.g., surfactants, film-forming polymers, solvents, fatty components, such as C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons; polymers; structurants, such as glucose, maleic acid, and lactic acid, hair-conditioning compounds, such as phospholipids, e.g., lecithin and cephalins; perfume oils, dimethyl isosorbide, and cyclodextrins; fiber structure-improving agents, and in particular mono-, di-, and oligosaccharides, e.g., glucose, galactose, fructose, and lactose; dyes for coloring the product; anti-dandruff active ingredients, such as piroctone olamine, zinc omadine, and climbazole; amino acids and oligopeptides; animal and/or vegetable-based protein hydrolyzates, as well as in the form of their fatty acid condensation products or optionally anionically or cationically modified derivatives; plant-based oils, light stabilizers, and UV blockers; active ingredients, such as panthenol, pantothenic acid, pantolactone, allantoin, pyrrolidinone carboxylic acids and the salts thereof, and bisabolol; polyphenols, and in particular hydroxycinnamic acids, 6,7-dihydroxycoumarins, hydroxybenzoic acids, catechins, tannins, leucoanthocyanidins, anthocyanidins, flavanones, flavones, and flavanols; ceramides or pseudoceramides; vitamins, provitamins, and vitamin precursors; plant extracts; fats and waxes, such as fatty alcohols, beeswax, montan wax, and paraffins; swelling agents and penetrants, such as glycerol, propylene glycol monoethyl ether, carbonates, hydrogen carbonates, guanidines, ureas, and primary, secondary, and tertiary phosphates; opacifiers, such as latex, styrene/PVP, and styrene/acrylamide copolymers; pearlescent agents, such as ethylene glycol mono- and distearate as well as PEG-3-distearate; and propellants, such as propane-butane mixtures, N₂O, dimethyl ether, CO₂, and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according to the invention preferably always in amounts of 0.0001 to 25 wt %, and in particular of 0.0005 to 15 wt %, relative to the total weight of the particular agent.

### Preparation of the Application Mixture by Mixing Agents (a) and (b) and Optionally (c)

In step (4) of the process according to the invention, a ready-to-use mixture is prepared by mixing agents (a) and (b). If a separately fabricated pigment is used in a third agent (c), a ready-to-use dye is prepared in step (4) of the process by mixing agents (a) and (b) and (c).

In principle, different amounts of the agent (a) can be mixed with the agent (b); thus, in principle, mixing ratios (a)/(b) of 1:400 to 400:1 are conceivable.

It is particularly preferred if the application mixture is prepared by mixing agents (a) and (b) in the weight ratio (a)/(b) of 1:1 to 1:100, preferably of 1:3 to 1:50, more preferably of 1:5 to 1:40, and even more preferably of 1:7 to 1:30.

In the case of a quantitative ratio (a)/(b) of 1:120, for example, 1 g of agent (a) can be mixed with 120 g of agent (b).

With a quantitative ratio (a)/(b) of 1:100, for example, 1 g of agent (a) can be mixed with 100 g of agent (b).

With a quantitative ratio (a)/(b) of 1:15, for example, 10 g of agent (a) can be mixed with 150 g of agent (b).

With a quantitative ratio (a)/(b) of 1:25, for example, 4 g of agent (a) can be mixed with 100 g of agent (b).

In the context of an additional preferred embodiment, a process according to the invention is characterized by
(3) preparing an application mixture by mixing agents (a) and (b) and optionally (c), wherein agents (a) and (b) are mixed with one another in a weight ratio (a)/(b) of 1:1 to 1:100, preferably of 1:3 to 1:50, more preferably of 1:5 to 1:40, even more preferably of 1:7 to 1:30, and very particularly preferably of 1:9 to 1:28.

### Application of the Application Mixture

In step (5) of the process according to the invention, the application mixture produced in step (4) is applied to the keratin fibers or hair.

Preferably, the application mixture is applied to the keratin material (or to the hair) within a period of 1 to 120 minutes, preferably 1 to 60 minutes, more preferably 1 to 30 minutes, and very particularly preferably 1 to 15 minutes after its production in step (4).

In a further preferred embodiment, a process according to the invention is characterized by
(5) applying application mixture to the keratinous material within a period of 1 to 120 minutes, preferably 1 to 60 minutes, more preferably 1 to 30 minutes, and very particularly preferably 1 to 15 minutes after its production in step (4).

### Allowing the Application Mixture to Act Upon the Keratin Material

In step (6) of the process according to the invention, the application mixture is then allowed to act upon the keratinous material after its application. In this context, various action times of, for example, 30 seconds to 60 minutes are conceivable.

However, a great advantage of the dyeing system according to the invention is that an intensive color result can be achieved even in very short periods after short action times. For this reason, it is advantageous for the application mixture to remain on the keratin material after application only for comparatively short periods of 30 seconds to 15 minutes, preferably 30 seconds to 10 minutes, and particularly preferably 1 to 5 minutes.

In a further preferred embodiment, a process according to the invention is characterized by
(6) allowing the application mixture applied in step (5) to act upon the keratinous material for a period from 30 seconds to 15 minutes, preferably from 30 seconds to 10 minutes, and particularly preferably from 1 to 5 minutes.

### Rinsing Out the Application Mixture

After the application mixture has been allowed to act upon the keratin material, the latter is lastly rinsed with water in step (7).

In one embodiment, the application mixture can be washed out with water only, i.e., without the aid of an after-treatment agent or a shampoo. The use of an after-treatment agent or conditioner in step (8) is also conceivable in principle.

### Sequence of Process Steps

The process according to the invention comprises steps (1) through (7) (or (1) through (6) if no third agent (c) is to be used).

In step (1), agent (a) is provided; step (2) comprises providing the agent (b).

In step (3), the optional agent (c) is provided.

These two or three steps do not necessarily have to take place in succession, but can also take place simultaneously.

Step (1) can thus take place before step (2), steps (1) and (2) can take place simultaneously, or step (2) takes place before step (1). Step (3) can take place before or after step (1) or (2). The steps (1), (2), and (3) also take place simultaneously.

If, for example, agents (a) and (b) and optionally (c) are made available to the user in a multi-component packaging unit, the agents are provided simultaneously, and it is left to the user which agent he removes first from the packaging.

The preparation of an application mixture by mixing the agents (a) and (b) and optionally (c) in step (4) can take place only after the preparation of agents (a) and (b) and optionally (c). The application mixture can be applied in step (5) only after it has been prepared in step (4). Analogously, the action of the application mixture in step (6) can be achieved only after application thereof to the keratin material, and rinsing out the application mixture in step (7) takes place after the action thereof in step (6).

### Multi-Component Packaging Unit

To increase convenience of use, the user is preferably provided with all the required agents in the form of a multi-component packaging unit (kit-of-parts).

A second subject matter of the present invention is therefore a multi-component packaging unit (kit-of-parts) for treating keratinous material, and in particular human hair, which comprises, fabricated separately from one another,
a first container having an agent (a), wherein agent (a) contains:
(a1) at least one amino-functionalized silicone polymer, and
(a2) at least one cosmetic oil which is liquid at 20° C. and is different from (a1), and
a second container having an agent (b), wherein agent (b) contains:
(b1) water and/or at least alkylene glycol of the formula (AG)

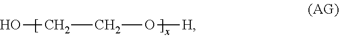

where
x represents an integer from 1 to 10,000, and
optionally a third container having an agent (c), wherein agent (c) contains:
(c1) at least one pigment,
wherein agents (a), (b), and (c) and components (a1), (a2), (b1), and (c1) are disclosed in detail in the description of the first subject matter of the invention.

The statements made about the process according to the invention apply mutatis mutandis with regard to the further preferred embodiments of the multi-component packaging unit according to the invention.

### Examples

### 1. Formulations

The following formulations were produced:

| | (aV) | (aE) |
|---|---|---|
| Agent (a) | Comparison | Invention |
| Dow Corning 2-8566 (siloxanes and | 0.75 g | 0.75 g |
| silicones, 3-[(2-aminoethyl)amino]-2- | | |
| methylpropyl Me, di-Me-siloxane] | | |
| Hexamethyldisiloxane (viscosity 0.65 cSt) | — | 6.75 g |
| Total amount of agent (a) | 0.75 g | 7.5 g |

| Agent (b) | (b) | |
|---|---|---|
| Water | 10.0 | g |
| PPG-3 benzyl ether myristate | 1.5 | g |
| Ucon Fluid AP | 1.0 | g |
| Phenoxyethanol | 0.2 | g |
| 4-hydroxyacetophenone | 0.2 | g |
| Unipure Red LC 3079 (pigment) | 1.0 | g |
| Polyethylene glycol (molecular weight | 10.0 | g |
| 6,000 g/mol) | | |
| Polyethylene glycol (molecular weight 400 | ad 100 g | |
| g/mol) | | |
| Total amount of agent (b) | 100 | g |

| Agent (c) | (c) |
|---|---|
| Silver pigment (Alegrace Marvelous D | 1.0 g |
| 12/77-1 Shiny Silver, 10%) | |
| Total amount of agent (c) | 1 g |

### 2. Preparation of the Application Mixture by Mixing (a) and (b)

Agent (b) was filled into an applicator bottle with tip. Thereafter, the respective agent (a) was added. The applicator bottle was provided with a sealed tip and shaken.

| AWM 1, comparison | AWM 2, invention |
|---|---|
| 0.75 g agent (aV) | 7.5 g agent (aE) |
| 100 g agent (b) | 100 g agent (b) |
| no mixing possible, since the | homogeneous mixture |
| aminosilicone settled in the applicator tip | |

### 3. Preparation of the Application Mixture by Mixing (a) and (b) and (c)

Agent (b) was filled into an applicator bottle with tip. Thereafter, the corresponding agent (a) was added. Agent (c) was then added.

The applicator bottle was provided with a sealed tip and shaken.

| AWM 3, comparison | AWM 4, invention |
|---|---|
| 0.75 g agent (aV) | 7.5 g agent (aE) |
| 100 g agent (b) | 100 g agent (b) |
| 1 g agent (c) | 1 g agent (c) |
| no mixing possible, since the | homogeneous mixture |
| aminosilicone settled in the applicator tip | |

What is claimed is:

1. A process for treating keratinous material, and in particular human hair, comprising the following steps:
(1) providing an agent (a), the agent (a) comprising:
(a1) at least one amino-functionalized silicone polymer, and
(a2) at least one cosmetic oil which is liquid at 20° C. and is different from (a1),
(2) providing an agent (b), the agent (b) comprising:
(b1) water and/or at least alkylene glycol of the formula (AG), 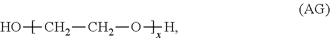
where
x is an integer from 1 to 10,000,
(3) optionally providing an agent (c), the agent (c) comprising:
(c1) at least one pigment,
(4) preparing an application mixture by mixing agents (a) and (b) and optionally (c),
(5) applying the application mixture prepared in step (4) to keratinous material,
(6) allowing the application mixture applied in step (5) to act upon the keratinous material, and
(7) optionally rinsing out the application mixture.

2. The process according to claim 1, wherein the agent (a) comprises at least one amino-functionalized silicone polymer (a1) having at least one secondary amino group.

3. The process according to claim 1, wherein the agent (a) comprises at least one amino-functionalized silicone polymer (a1) comprising at least one structural unit of the formula (Si-amino), 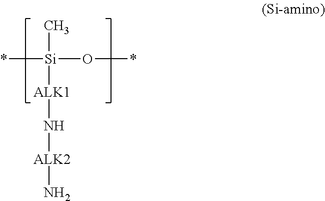
where
ALK1 and ALK2 represent, independently of one another, a linear or branched, divalent C₁-C₂₀ alkylene group.

4. The process according to claim 1, wherein the agent (a) comprises at least one amino-functionalized silicone polymer (a1) comprising structural units of the formula (Si—I) and of the formula (Si-II)

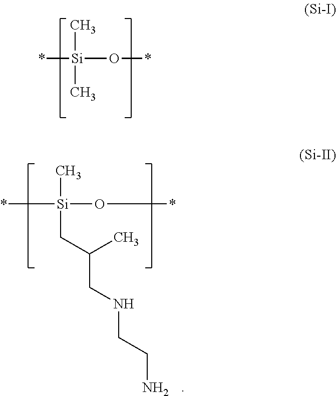

5. The process according to claim 1, wherein the agent (a) comprises, relative to the total weight of agent (a), one or more amino-functionalized silicone polymers (a1) in a total amount of 1 to 90 wt %.

6. The process according to claim 1, wherein the agent (a) comprises, relative to the total weight of agent (a), one or more amino-functionalized silicone polymers (a1) in a total amount of 5 to 20 wt %.

7. The process according to claim 1, wherein the agent (a) comprises at least one cosmetic oil (a2), which is liquid at 20° C. and is selected from the group consisting of non-amino-functionalized oligoalkylsiloxanes, non-amino-functionalized silicone polymers, paraffin oils, isoparaffin oils, synthetic C₃-C₁₂ hydrocarbons, di-C₁₂-C₂₃ alkyl ethers, vegetable oils, and ester oils.

8. The process according to claim 1, wherein the agent (a) comprises at least one cosmetic oil (a2), which is liquid at 20° C. and is selected from the group consisting of oligoalkylsiloxanes of the formula (OAS-I) and/or (OAS-II), 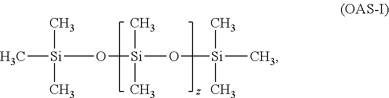
where z represents an integer from 0 to 10, 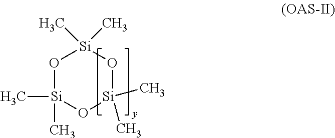
where y represents an integer from 1 to 5.

9. The process according to claim 1, wherein the agent (a) contains at least one cosmetic oil (a2), which is liquid at 20° C. and is selected from the group consisting of polydimethylsiloxanes and has a viscosity of 0.5 to 30,000 mm²/s, measured according to ASTM Standard D-445.

10. The process according to claim 1, wherein the agent (a) comprises at least one cosmetic oil (a2), which is liquid at 20° C. and is selected from the group consisting of polydimethylsiloxanes and has a viscosity of 0.5 to 500 mm²/s, measured according to ASTM Standard D-445.

11. The process according to claim 1, wherein the agent (a) comprises, relative to the total weight of agent (a), one or more cosmetic oils, which are liquid at 20° C., in a total amount of 1 to 99 wt %.

12. The process according to claim 1, wherein the agent (a) comprises the cosmetic oils (a2) and the amino-functionalized silicone polymers (a1) at a weight ratio ((a2)/(a1)) of 100:1 to 1:100.

13. The process according to claim 1, wherein the agent (a) comprises the cosmetic oils (a2) and the amino-functionalized silicone polymers (a1) at a weight ratio ((a2)/(a1)) of 20:1 to 4:1.

14. The process according to claim 1, wherein the agent (b) comprises, relative to the total weight of agent (b), one or more alkylene glycols (b1) of the formula (AG) in a total amount of 10.0 to 99.0 wt %.

15. The process according to claim 1, wherein the agent (b) comprises (b2) at least one pigment.

16. The process according to claim 1, further comprising the steps of
(3) providing an agent (c), the agent (c) comprising:
(c1) at least one pigment, and
(4) preparing an application mixture by mixing agents (a) and (b) and (c).

17. The process according to claim 1, wherein the agent (b) comprises:
(b3) at least one alkoxylated fatty acid ester of the general formula (AFE-I) 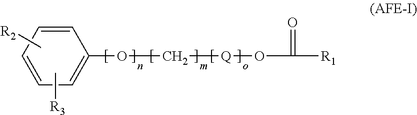
where
R1 represents a saturated or unsaturated C₁₁-C₂₉ alkyl group,
R2, R3 represent, independently of one another, a hydrogen atom, a C₁-C₆ alkyl group, a hydroxy group, or a C₁-C₆ alkoxy group,
n represents the number 0 or 1,
m represents an integer from 0 to 6,
o represents an integer from 1 to 60, and
Q represents a structural unit —O—CH₂—CH₂—, —O—CH(CH₃)—CH₂—, or —O—CH₂—CH(CH₃)—
with the proviso that if m is equal to 0, n is also equal to 0.

18. The process according to claim 1, wherein the agent (b) comprises:
(b4) at least one addition product of C₁-C₆ alkylene oxide(s) to aliphatic C₁-C₂₄ alkanols of formula (AA-J), 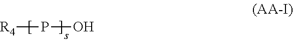
wherein
R4 represents a saturated or unsaturated C₁-C₂₄ alkyl group, and
P represents a structural unit —O—CH₂—CH₂—, —O—CH(CH₃)—CH₂—, or —O—CH₂—CH(CH₃)—, and
s represents an integer from 1 to 60.

19. The process according to claim 1, wherein agents (a) and (b) are mixed with one another in a weight ratio (a)/(b) of 1:1 to 1:100.

20. A multi-component packaging unit (kit-of-parts) for treating keratinous material, and in particular human hair, comprising, fabricated separately from one another,
a first container having an agent (a), wherein the agent (a) comprises:
(a1) at least one amino-functionalized silicone polymer, and
(a2) at least one cosmetic oil which is liquid at 20° C. and is different from (a1), and
a second container having an agent (b), wherein agent (b) comprises:
(b1) water and/or at least alkylene glycol of the formula (AG) 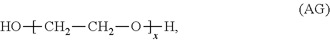
where
x is an integer from 1 to 10,000, and
optionally a third container having an agent (c), wherein agent (c) contains:
(c1) at least one pigment.
